(19) United States
(12) Patent Application Publication
(10) Pub. No.: US 2003/0023247 A1
(43) Pub. Date: Jan. 30, 2003

(54) Medical retrieval device with cable protection means

(76) Inventors: Stuart J. Lind, Edina, MN (US); Mark R. Bilitz, Plymouth, MN (US)

Correspondence Address: ANNEX MEDICAL, INC., 6018 BLUE CIRCLE DRIVE, MINNETONKA, MN, 55343, US

(21) Appl. No.: 10/189,858

(22) Filed: Jul. 3, 2002

Related U.S. Application Data
(60) Provisional application No. 60/303,000, filed on Jul. 3, 2001.

(51) Int. Cl. A61B 17/22

(52) U.S. Cl. 606/127

(57) ABSTRACT

A medical retrieval device used to remove objects such as urinary and biliary calculi from the body comprising a handle assembly with a cable and a cable protection means. In a preferred embodiment, the handle assembly comprises a longitudinally stationary base portion and a thumb slide actuation means. A portion of the thumb slide actuation means is located between the distal and proximal ends of the handle base portion during the complete actuation cycle. Actuation of the thumb slide relative to the handle base portion causes the retrieval device to be actuated between the open and closed positions. The handle is preferably sized and configured to be held and operated comfortably with one hand. The light weight of the entire handle assembly minimizes potential damage to the small diameter flexible cable. In preferred embodiments, a series of openings in the handle base reduce weight, reduce material usage, and facilitate sterilization while maintaining strength. In a preferred embodiment, the cable extends out of a hole in the base portion in a loose fit arrangement, allowing radial movement of the cable by at least 0.5 mm (or 50% of the sheath diameter) relative to the base portion.

This application claims priority to U.S. Provisional Application No. 60/303,000 filed Jul. 3, 2001, which is incorporated herein by reference in its entirety. This application is also related to U.S. design Pat. No. D457,955 S, issued May 28, 2002, invented by Mark Bilitz. Said patent is incorporated herein by reference.

### BACKGROUND

1. Field of Invention

This invention relates to medical retrieval devices for removing objects from a body, particularly calculi from the urinary and biliary systems.

2. Description of Prior Art

Medical instruments are currently in use which reduce the invasiveness and potential trauma previously associated with various medical procedures. One such procedure is the removal of objects, such as kidney stones and gallstones, from the body. Various surgical devices are available which allow objects to be removed from the body without requiring major surgery. One type of surgical device is a mechanical retrieval device. Typically, such instruments consist of 2 or more flexible elements that are joined at their proximal ends and may or may not be joined at their distal ends. The flexible elements are formed in the shape of a basket, cage, grasper, or other entrapping configuration. This basket is attached to a drive wire that passes through the lumen of a small diameter (typically 2.3 mm (7.0 Fr) or less) flexible sheath, which is usually greater than 50 cm in length. The proximal ends of the sheath and drive wire are attached to a multi-part handle, normally constructed of thermoplastic materials, which can typically be operated by the user with a single hand. By manipulating the handle, the drive wire can be pulled back relative to the sheath, collapsing the basket as it retracts into the sheath. In this closed position, the sheath can be passed through the working channel of an endoscope to the proximity of the object to be removed within the patient's body. The basket is expanded to the open position by manipulating the handle, which remains outside the endoscope and the patient's body. The device is then manipulated using the handle until the object becomes enclosed within the basket. The endoscope and the retrieval device containing the object are then simultaneously removed from the body.

A number of designs for the handle of medical retrieval devices are in use. U.S. Pat. No. 1,677,671 (1928) to Councill discloses a retrieval basket with a handle that is held and operated using three rings. This handle consists of two main elements. The first element is connected to the sheath and has a ring at the opposite end. The second element is connected to the drive wire and has two rings. The second element attaches to the first element and can slide along its middle section. This type of handle is held by placing the thumb of one hand through the ring in the first element, and the first two fingers of the same hand through the two rings of the second element. The device is actuated by drawing the two fingers toward the thumb and extending them away from the thumb. A similar type of handle is shown in U.S. Pat. No. 6,090,129 (2000) to Ouchi (especially in FIGS. 4 and 5). Instead of a second element with two rings, it uses a second element that is spool-shaped and fits around the shaft of the first element. This type of handle is held by placing the thumb of one hand through the ring in the first element, and grasping the spool-shaped element with the first two fingers of the same hand, one on each side of the spool. The device is actuated by drawing the two fingers toward the thumb and extending them away from the thumb. Both of these handle designs have the disadvantage that the user's thumb and fingers must be held in somewhat unnatural positions for the entire duration that the device is being used. Since considerable skill and dexterity can be required of the user in order to retrieve an object, user comfort is of primary importance. While these handle designs have been used for many years, they have been widely replaced recently with other designs.

U.S. Pat. No. 4,046,150 (1977) to Schwartz et al. discloses a retrieval basket with another type of handle. This handle has a first member that is attached to the sheath. A second member, which is attached to the drive wire, is located at the proximal end of the first member. This type of device is actuated by sliding the second member into and out of the first member, which is held stationary. This handle design has the disadvantage that it is difficult to operate with one hand.

U.S. Pat. No. 5,957,932 (1999) to Bates et al. discloses a retrieval basket with yet another type of handle. This handle is of a pistol grip style, with a control knob located at the proximal end of the handle. The sheath is attached to the main body of the handle, and the drive wire is attached to the control knob. The device is actuated by pulling the control knob out from the main body of the handle and pushing it in. The control knob can also be rotated to rotate the basket. When the trigger portion of the handle is squeezed, mechanical advantage is applied to the actuation of the device. This design has several disadvantages. It requires two hands for normal actuation. It also uses a larger number of parts than other handle designs and is therefore is more complex and more expensive to manufacture. The above patents to Councill, Schwartz, and Bates are incorporated herein by reference.

Another type of prior art handle, which is not referenced in any patents, is shown in FIGS. 7 and 8. A handle assembly 190 consists of a stationary portion or handle base 110 and a thumb slide 130 with a thumb pad 131. Handle base 110 has a distal end 181, a proximal end 182, and a length 180. Thumb slide 130 fits partially within handle base 110 and extends out from distal end 181, with thumb pad 131 remaining beyond distal end 181. A hollow tube or sheath 150 has a working length 186 and is attached to thumb slide 130. A drive wire (not shown) passes through the lumen of sheath 150 attaches at the proximal end to handle base 110 and at the distal end to a basket 160. Sheath 150 and the drive wire (not shown) together comprise a cable 175. To operate this device, handle base 110 is held in one hand with the four fingers of the hand. The thumb of the same hand is placed on thumb pad 131. When the thumb is extended away from the hand, thumb slide 130 slides out from distal end 181 of handle base 110. This results in the device being in the closed position, as shown in FIG. 7. When the thumb is pulled back toward the hand, thumb pad 131 slides toward distal end 181 of handle base 110. This results in sheath 150 being pulled back to expose basket 160. When thumb pad 131 is pulled back completely to handle base 110, the device is in the open position, as shown in FIG. 8. This design has the disadvantage that the thumb pad is located beyond the distal end of the stationary handle base. This is a disadvantage because manipulation of the thumb slide is done with the user's thumb extended away from the hand in a somewhat awkward position, which results in less than ideal tactile control over the actuation of the device.

All of the prior art handle designs discussed to this point have been used for medical retrieval devices. However, the design that appears to be preferred, based on actual current use and sales volumes, is of another thumb slide design. This design features a main handle base that remains stationary and a thumb slide actuator that slides along a portion of the handle base and has a thumb pad. The area of travel of at least a portion of the thumb pad is located between the distal and proximal ends of the stationary handle base. This handle is held in one hand by wrapping the four fingers of the hand partially around the handle base. The thumb of the same hand is placed on the thumb pad. The device is actuated in one direction by moving the thumb pad away from the proximal end of the handle base, and in the other direction by moving the thumb pad toward the proximal end of the handle base. This type of handle can normally be held in such a way that the range of motion of the thumb required to fully actuate the device is located in a natural and comfortable area near the thumb's resting position. A number of variations of this type of thumb slide handle have been used. The following U.S. patents disclose medical retrieval devices that use handles with some of these variations. The figures from these prior art patents that specifically show the handle are indicated.

U.S. Pat. No. 5,496,330 (1996) to Bates et al. (FIGS. 6, 7, and 8)

U.S. Pat. No. 5,573,530 (1996) to Fleury et al. (FIGS. 6, 7, and 8)

U.S. Pat. No. 5,817,104 (1998) to Bilitz et al. (FIGS. 10, 11A, 11B, and 11C)

U.S. Pat. No. 6,096,053 (2000) to Bates (FIG. 10A)

These and other similar prior art designs all have the disadvantage of significant handle weight. As technology has improved, endoscopes have been developed which have smaller diameters, allowing them to become less invasive. In order to make this possible, the sheath diameters of retrieval devices have also been reduced. Many retrieval devices in use today for urinary and biliary calculi have sheath diameters of approximately 1 millimeter or less. This has resulted in the cable portion of the device, consisting of the sheath and drive wire, being quite delicate and susceptible to damage.

While sheath diameters have been decreasing, making them more delicate and susceptible to damage, handles have generally not seen a reduction in size or weight. In fact, several commercially available handles have become heavier and more complex as attempts have been made to add new features.

The majority of the weight of these devices is in the handle assembly. Excessive weight of the handle assembly can contribute to causing the cable (sheath and drive wire) to kink. For example, during a retrieval procedure, the device is inserted into the endoscope so that only the handle and proximal portion of the cable remain outside of the endoscope. If it is necessary for the user to let go of the handle in this position, for example, to adjust the endoscope, the handle would dangle from the endoscope. A handle of significant weight, combined with a delicate cable, can cause a bend or kink in the cable at the exit point of the cable from the endoscope. In the worst case situation, damage to the sheath and/or drive wire could occur during a procedure, causing complications and possible injury to the patient. Also, even if the cable is not damaged, a handle assembly of significant weight dangling from the endoscope could cause an undesirable repositioning of the basket within the patient, lengthening the procedure.

Moreover, dropping a handle assembly where the sheath adjacent to the handle assembly impacts the floor with the weight of the handle assembly behind the sheath can cause a bend or kink at the exit point of the sheath from the handle assembly.

FIG. 9 shows the weight of some of the preferred prior art handles; that is to say, handles that use thumb slide actuation and for which the area of travel of at least a portion of the thumb pad is located between the distal and proximal ends of the handle base. For this comparison, the weight of the handle assembly is defined as the weight of all components proximal to the most proximal point of the exposed sheath that occurs distally of all handle assembly components. The handle assembly is assumed to include luer connectors as shown in U.S. Pat. No. 6,096,053 and other detachable components as shown in U.S. Pat. No. 5,496,330. These patents are incorporated herein by reference. Another prior art handle of this preferred type, which is not shown in FIG. 9, is a handle used for products previously marketed under the trade name Van-Tec. This handle had an approximate weight of 10 grams. However, this handle had the disadvantage of a stationary portion which is shorter (approximately 8.6 cm in length) and narrower (approximately 1 cm in width), resulting in a less comfortable fit in the user's hand than handles with stationary portions that are longer (10 cm or greater) and/or wider (1.2 cm or greater). This handle was replaced many years ago by one or both of the first two handles listed in FIG. 9.

Generally, the prior art devices have the sheath fixed to the distal end of the handle assembly or have the sheath extending out of a hole sized to a close tolerance to the size of the sheath. Such arrangements make the cable at the exit point of the handle portion particularly susceptible to damage.

Similarly, during handling, transit, and storage, prior art devices must be restrained in some way within the sterile package to prevent the weight of the handle from damaging the sheath and drive wire. This is normally accomplished using plastic thermoformed trays as protective packaging. While this has become normal practice, it adds additional cost and waste to the product.

Attempts have been made to reduce the risk of damaging the sheath and drive wire at the outlet of the handle assembly by providing a tapered or staged strain relief at the outlet to distribute any bending forces over greater length of the sheath and drive wire thus minimizing the potential for bends and kinks. This requires complex or multiple components, which increases the amount of material, the weight, and the complexity of assembly, increasing manufacturing costs.

Generally, the prior art designs utilize handle assemblies that encase or otherwise conceal internal moving parts. This can make the sterilization and aeration of the device by ethylene oxide, steam, or other gaseous sterilization means more complex by reason of the concealed components.

### SUMMARY OF THE INVENTION

A medical retrieval device used to remove objects such as urinary and biliary calculi from the body comprising a handle assembly with a cable and a cable protection means. In a preferred embodiment, the handle assembly comprises a longitudinally stationary base portion and a thumb slide actuation means. A portion of the thumb slide actuation means is located between the distal and proximal ends of the handle base portion during the complete actuation cycle. Actuation of the thumb slide relative to the handle base portion causes the retrieval device to be actuated between the open and closed positions. The handle is preferably sized and configured to be held and operated comfortably with one hand. The light weight of the entire handle assembly minimizes potential damage to the small diameter flexible cable. In preferred embodiments, a series of openings in the handle base reduce weight, reduce material usage, and facilitate sterilization while maintaining strength. In a preferred embodiment, the cable extends out of a hole in the base portion in a loose fit arrangement, allowing radial movement of the cable by at least 0.5 mm (or 50% of the sheath diameter) relative to the base portion.

An object and advantage of preferred embodiments of the invention is to provide a novel handle for a medical retrieval device of a thumb slide actuated type that is comfortable for the user to hold and actuate.

An object and advantage of preferred embodiments is that the handle for a medical retrieval device is of lighter weight (7 grams or less) than prior art designs (12 to 33 grams). This reduces the likelihood of inadvertent damage to the small diameter flexible cable (consisting of the sheath and drive wire) of the device, and also reduces the likelihood of the weight of the handle inadvertently causing repositioning of the device within the patient.

An object and advantage of preferred embodiments is a medical retrieval device for which the delicate cable extends out of a hole in the handle assembly in a loose fit arrangement with the hole being significantly larger diametrically than the cable, further protecting the cable from damage.

An object and advantage of preferred embodiments is a handle for a medical retrieval device that requires less protective packaging than prior art designs for preventing damage to the small diameter flexible sheath and drive wire during transit and handling.

An object and advantage of preferred embodiments is a medical retrieval device having a handle that is strong and durable and can withstand the forces applied to it during use while still providing the other advantages identified herein.

An object and advantage of preferred embodiments is to provide a novel handle for a medical retrieval device that requires a minimum amount of material resulting in a part cost reduction.

An object and advantage of preferred embodiments is to provide a novel handle for a medical retrieval device that uses a minimal number of parts, reducing cost and simplifying assembly.

An object and advantage of preferred embodiments is to provide a novel handle for a medical retrieval device that is more readily sterilized by ethylene oxide, steam, or other gaseous sterilization means than prior art handle assemblies.

A further object and advantage of preferred embodiments is that the handle assembly is easier to grip with multiple apertures or openings providing gripping structure for minimizing slippage of the device in a hand during use.

A further object and advantage of preferred embodiments is that for a given size handle, the weight can be reduced over prior art handles.

Further objects and advantages of preferred embodiments of the medical retrieval device described herein are that such preferred embodiments are safe, reliable, and easy to use. Still further objects and advantages will become apparent from a consideration of the ensuing description and drawings.

### BRIEF DESCRIPTION OF THE DRAWINGS

FIG. 1 is an isometric view of the preferred embodiment of the present invention.

FIG. 2 is an exploded isometric view of FIG. 1.

FIG. 3 is a top view of FIG. 1 showing the basket in the retracted, closed, or collapsed position.

FIG. 4 is a top view of FIG. 1 showing the basket in the extended, open, or expanded position.

FIG. 5 is a bottom view of FIG. 1 showing the basket in the extended, open, or expanded position.

FIG. 5A is a cross sectional view of FIG. 5 taken on line A-A.

FIG. 5B is a cross sectional view of FIG. 5 taken on line B-B.

FIG. 6 is a side view, partially in section along the longitudinal axis, of the handle assembly portion of the medical retrieval device in FIG. 1.

FIG. 6A is an enlarged view of the proximal portion of the handle assembly in FIG. 6.

FIG. 6B is an enlarged view of the mid portion of the handle assembly in FIG. 6.

FIG. 6C is an enlarged view of the distal portion of the handle assembly in FIG. 6.

FIG. 7 is side view of a prior art medical retrieval device showing the basket in a retracted position, the top view and bottom view being the same.

FIG. 8 is a side view of the same prior art device in FIG. 7 but showing the basket in an extended position, the top view and bottom view being the same.

FIG. 9 is a table showing a comparison of some currently marketed stone retrieval devices with thumb slide handles having thumb pads that overlap the stationary portion.

### DETAILED DESCRIPTION OF PREFERRED EMBODIMENTS

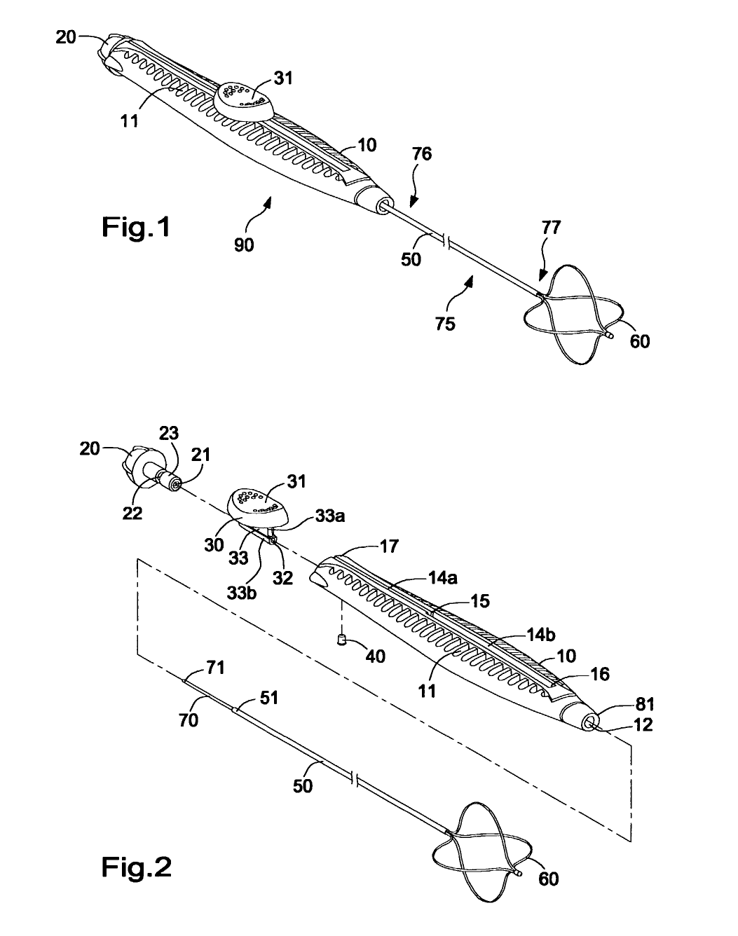

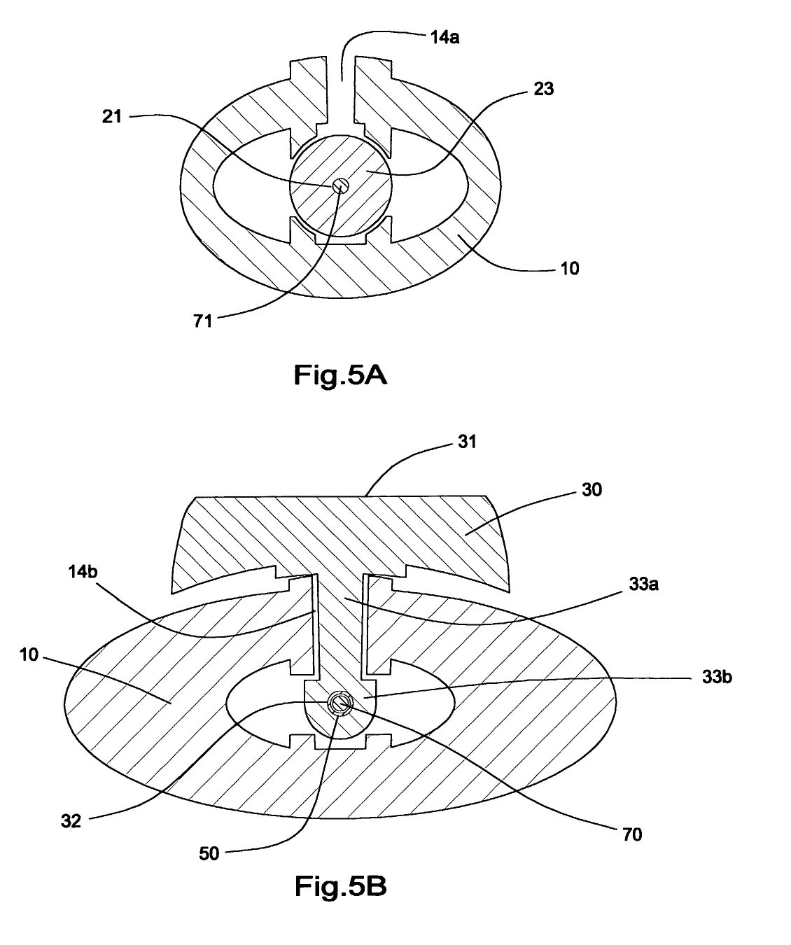

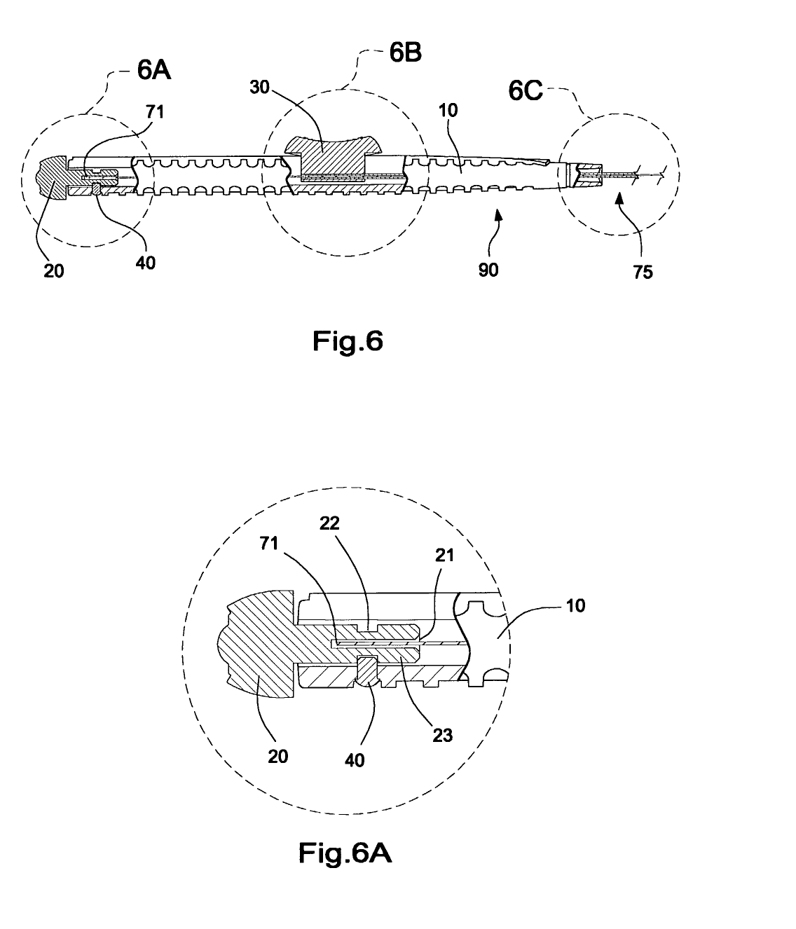

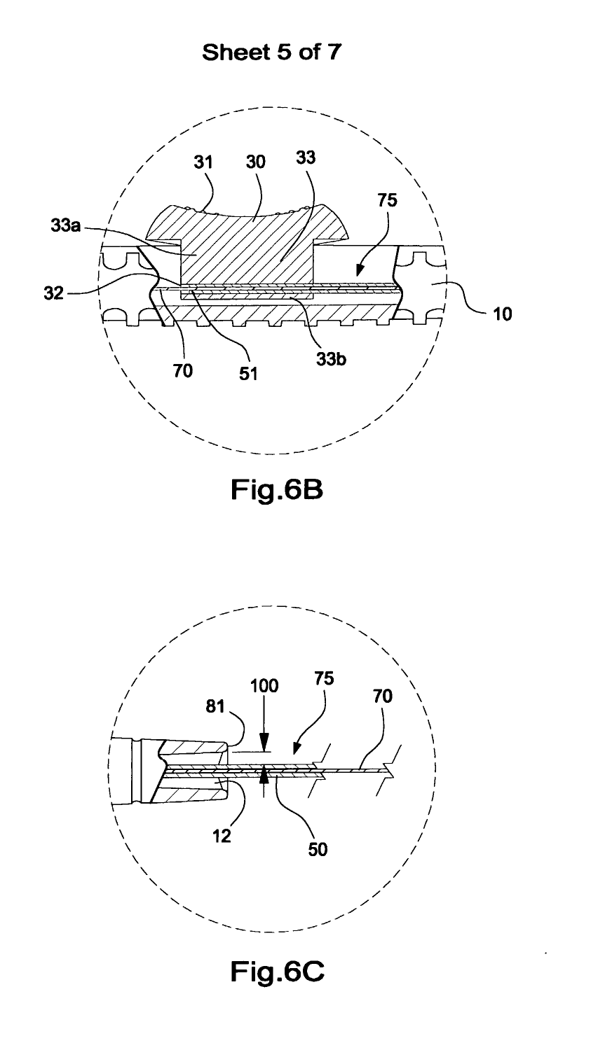

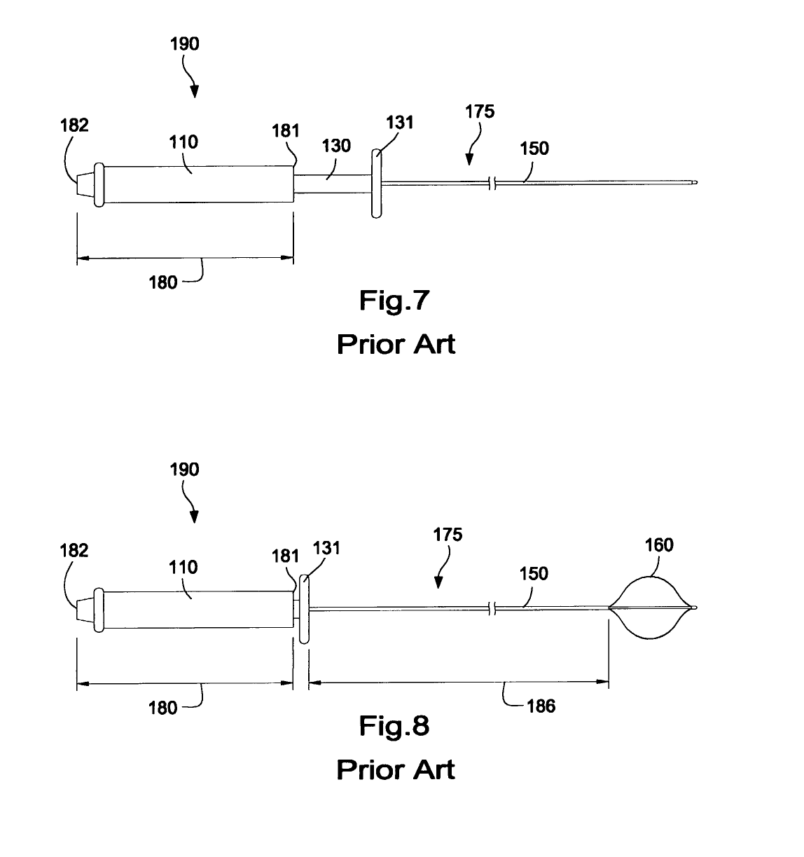

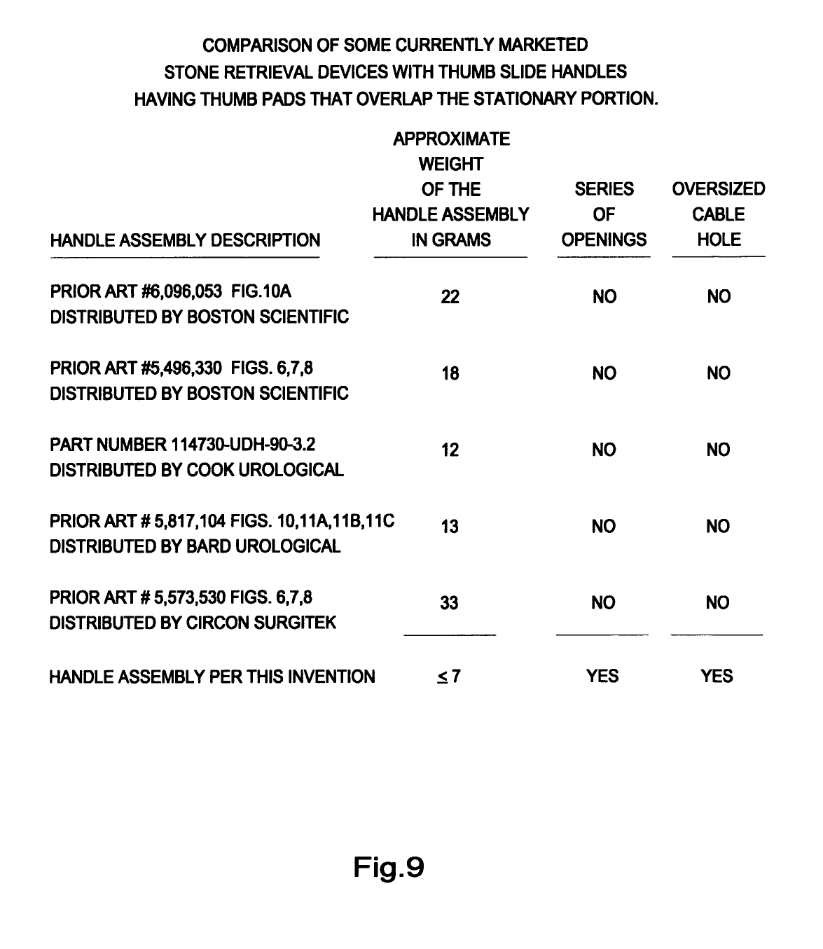

FIGS. 1 to 6C show a preferred embodiment of the invention. A medical retrieval device has an actuation means comprising a handle assembly 90. Handle assembly 90 is comprised of a handle base 10, an end cap 20, a sliding portion or thumb slide 30, and a pin 40, all of which are constructed of thermoplastic materials, without the use of foaming agents. Thermoplastics typically used for disposable medical devices have specific gravity ranging from 0.9 to 1.5. Handle base 10 has a proximal end 17 and a distal end 81. Handle base 10 is essentially hollow along its longitudinal axis, and has a hole 12 at distal end 81. A slot having a narrow portion 14a and a wide portion 14b is located on the top surface of handle base 10. Wide portion 14b has a proximal end 15 and a distal end 16. Narrow portion 14a extends from proximal end 17 of handle base 10 to wide portion 14b. Distal end 16 of wide portion 14b is located proximal to distal end 81 of handle base 10. Two series of openings 11 may extend longitudinally down handle base 10, reducing the weight of handle assembly 90. At least ten openings 11 are preferably present in handle base 10. The openings may extend completely through handle base 10 or partially through. Other means may be used to reduce the weight such as adding foaming agents to add some porosity.

Thumb slide 30 has a thumb pad 31 and a guide portion configured as a fin 33, which has an upper portion 33a and a lower portion 33b. The width of upper portion 33a of fin 33 is wider than the width of narrow portion of slot 14a and narrower than the width of wide portion of slot 14b. The width of lower portion 33b of fin 33 is wider than the width of wide portion of slot 14b. A through hole 32 passes through lower portion 33b of fin 33 longitudinally. Thumb slide 30 is located on handle base 10 with upper portion 33a of fin 33 located within wide portion of slot 14b and lower portion 33b of fin 33 located within the hollow portion of handle base 10 below wide portion 14b. In this location, thumb slide 30 can be readily moved so that fin 33 is in any position within wide portion of slot 14b between proximal end 15 and distal end 16, but cannot inadvertently be removed vertically or longitudinally from wide portion of slot 14b in handle base 10. A tube or sheath 50 has a proximal end 51, and may be constructed of polyimide, PTFE, or other flexible material or combination of materials, and has a lumen through its entire length. Sheath 50 has a working length 86 that is between 65 and 125 cm, and the diameter of sheath 50 is preferably approximately 1 mm or less. Sheath 50 passes through hole 12, which is substantially larger in diameter than sheath 50, allowing radial movement 100 of sheath 50 by at least 0.5 mm (for a 1 mm sheath) relative to handle base 10. Proximal end 51 of sheath 50 passes into through hole 32 in thumb slide 30 and is secured to thumb slide 30 using adhesive or other means.

End cap 20 has a cylindrical portion 23 that has a groove 22 extending around its circumference. A hole 21 is located along the longitudinal axis of cylindrical portion 23, and extends part way through end cap 20. End cap 20 is located at proximal end 17 of handle base 10, with cylindrical portion 23 located within the hollow space of handle base 10. Pin 40 fits snugly into a hole in handle base 10, and a portion of pin 40 fits into groove 22 to secure end cap 20 to handle base 10. End cap 20 may or may not rotate about the longitudinal axis in relation to handle base 10. A drive wire 70 has a proximal end 71 and has an object entrapping assembly or basket 60 at its opposite end. Basket 60 consists of a number of outwardly disposed flexible elements that form a space for entrapping objects. Drive wire 70 may be constructed of stainless steel, nickel titanium alloy, or another metal. Drive wire 70 passes through the lumen of sheath 50, with proximal end 71 of drive wire 70 extending past proximal end 51 of sheath 50. Proximal end 71 of drive wire 70 fits into hole 21 in end cap 20 and is secured using adhesive or other means. Sheath 50 and drive wire 70 together comprise a cable 75, which has a proximal end 76 and a distal end 77. Handle assembly 90 has a longitudinally stationary portion which has a length 80 measured from a proximal end 82 to distal end 81, has a width 79, and comprises handle base 10, end cap 20, and pin 40. Length 80 is 10 cm or greater, and width 79 is 1.2 cm or greater. Thumb pad 31 is located between proximal end 82 and distal end 81. The actuation of thumb pad 31 overlaps the area between proximal end 82 and distal end 81.

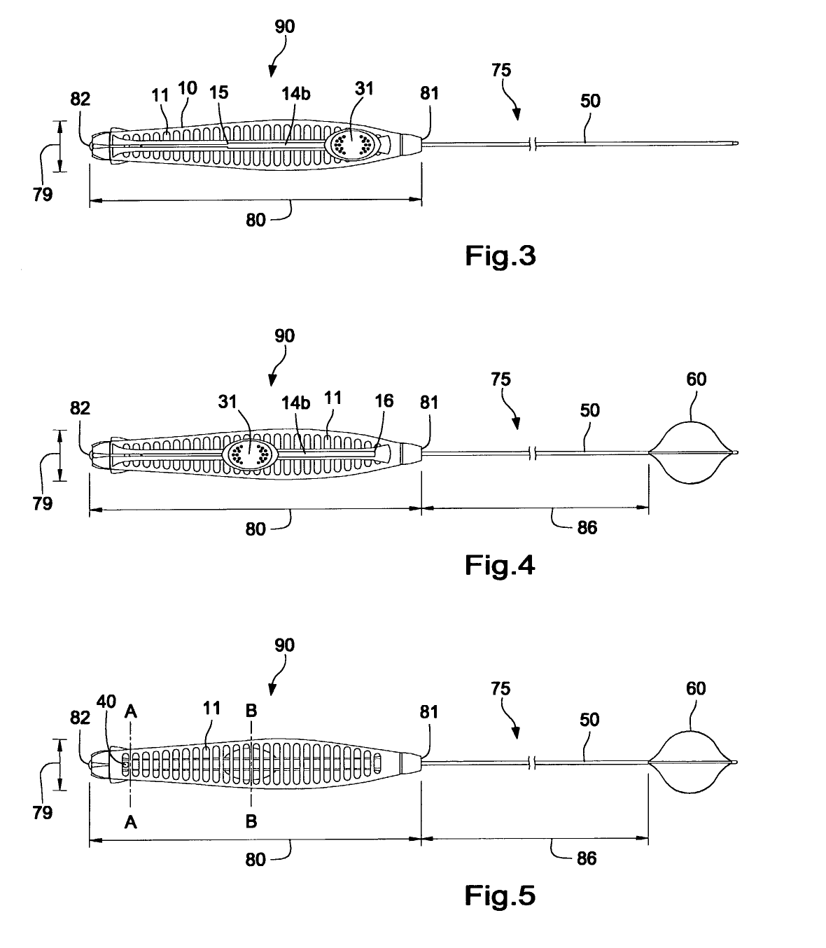

To operate the device, the user wraps four fingers of one hand partially around handle base 10, but not overlapping thumb pad 31. The tip of the thumb of the same hand is placed on thumb pad 31. Referring to FIG. 4, basket 60 is in the open or expanded position. In this position, thumb pad 31 is located at proximal end 15 of wide portion of slot 14b. To actuate the device to the closed or retracted position, the user extends the thumb outward from the hand while maintaining contact between the thumb and thumb pad 31 and keeping handle base 10 stationary in the hand. This action causes thumb slide 30 to slide within wide portion of slot 14b toward distal end 16 of wide portion of slot 14b. This propels sheath 50 over drive wire 70, forcing basket 60 to collapse and become enclosed within sheath 50. When thumb slide 30 rests at distal end 16 of wide portion of slot 14b, basket 60 is in the completely closed position within sheath 50, as shown in FIG. 3. If, for example, the object the device is being used to retrieve is a ureteral calculus, the device is introduced in this retracted position through the working channel of an endoscope into the ureter until the retracted basket emerges from the tip of the endoscope in the proximity of the calculus. The user then pulls the thumb back toward the hand, sliding thumb pad 31 back toward proximal end 15 of wide portion of slot 14b. This action pulls sheath 50 back relative to drive wire 70 and basket 60. Basket 60 is then exposed and resumes its expanded shape, as shown in FIG. 4. Handle assembly 90 is then manipulated until the object becomes entrapped within basket 60. The user then pushes thumb pad 31 forward part way toward distal end 16 of wide portion of slot 14b until basket 60 is partially closed in order to securely hold the object. The device and the endoscope are then simultaneously withdrawn from the patient, holding thumb pad 31 stationary relative to handle base 10 to keep the object secured within basket 60.

It can be seen from FIG. 9 and from the above description that the handle assembly according to this invention is of significantly less weight (7 grams or less) than the prior art designs (typically 12 to 33 grams). The weight of preferred embodiments is between 2 and 6 grams. In an alternate embodiment the weight is less than 5 grams. This reduces the possibility of the weight of the handle inadvertently causing damage to the delicate cable portion (sheath and drive wire) of the device. This also reduces the possibility of the weight of the handle inadvertently causing the device to be repositioned during a procedure. This is accomplished using a unique handle assembly design that requires less material than prior art designs, without compromising the size or strength of the handle assembly. This design includes moving material away from the longitudinal axis of the handle base, and providing a plurality of openings in the handle base. The space or area that is provided by each opening is preferably 2 cm² or less. None of the prior art handles of the preferred thumb slide design known to the inventors, such as those listed in FIG. 9, have openings 2 cm² or less. It can also be seen that the handle assembly according to this invention is comfortable to use. In the preferred embodiments, this is accomplished by keeping the length of the longitudinally stationary portion 10 cm or greater, similar to that of the preferred prior art designs currently in use. The length is significant because a lightweight handle could obviously be made by making it shorter. However, this would not be as comfortable for the user to hold and operate. Length of about 10 cm or greater is needed for a comfortable design. In a similar manner, a very thin handle could be made, but wouldn't satisfy the ergonomic requirements. For the preferred embodiments, at least 25% of the length of the longitudinally stationary portion has a width of 1.2 cm or greater. Additionally, a handle could be made similar to current prior art designs using lighter material, but the strength and durability of the handle may be compromised.

It can also be seen that as a result of the significantly reduced handle weight, the protective packaging used within the sterile package to prevent the handle from damaging the cable during transit can be reduced or eliminated. It can also be seen for the preferred embodiments that since the cable extends out of an oversized hole in the handle base in a loose fit arrangement, the handle assembly provides further protection for the cable. None of the prior art handles of the preferred thumb slide design known to the inventors, such as those listed in FIG. 9, have an oversized cable hole. It can also be seen that the handle's open design enhances the circulation and aeration of ethylene oxide, steam, and other gaseous sterilization means. It can further be seen that this is accomplished without using a large number of components to construct the handle assembly. This gives the desirable result of a handle assembly of a preferred style which is comfortable to hold and actuate, but is lightweight to reduce the possibility of causing damage to the delicate sheath and drive wire during handling and use.

Accordingly, the reader will see that the preferred embodiments of the present invention have a handle assembly of a thumb slide actuated type that is comfortable for the user to hold and actuate and is lightweight, reducing the possibility of inadvertently causing damage to the delicate cable portion of the device. In addition to serving as a cable protection means, the light weight of the handle assembly also reduces the possibility of inadvertently causing the device to be repositioned. Furthermore, the preferred embodiments have the additional advantages in that they have a handle assembly that

has a loose fit arrangement at the point where the cable emerges from the handle base, which provides further cable protection means;

requires less protective packaging than prior art designs for preventing damage to the small diameter flexible sheath and drive wire during transit and handling;

is strong and durable and can withstand the forces applied to it during use, while still providing the other advantages identified herein;

requires a minimum amount of material resulting in a part cost reduction;

uses a minimal number of parts, reducing cost and simplifying assembly;

is more readily sterilized by ethylene oxide, steam, or other gaseous sterilization means than prior art handles; and

is easier to grip with multiple aligned openings providing a gripping structure for minimizing slippage of the device in a hand during usage.

Another aspect of the invention is the method of manufacturing the medical retrieval device as described above. More specifically, the method involves steps of injection molding a handle with the above characteristics and assembling the device whereby the device has a cable protection means.

Although the description above contains many specificities, these should not be construed as limiting the scope of the invention, but as merely providing illustrations of some of the presently preferred embodiments of this invention. For example, the handle base might not be longitudinally hollow; the openings may not all pass completely through the handle base; the number and arrangement of the openings in the handle base may vary; the direction the thumb slide is actuated to open the basket may be reversed; the sheath may be attached to the handle base or the end cap; the drive wire may be attached to the thumb slide or the handle base; the sheath and/or drive wire may be removable from the handle assembly; a coaxial, dual sheath design may be used; additional and/or alternative materials and joining methods may be used; the handle assembly components may have other configurations; other means may be used to control the distance of travel of the thumb slide; the end cap and/or pin may be eliminated; the thumb pad may only partially overlap the handle base at part of its travel; the thumb slide and handle base may fit together by different means; the basket may have other configurations; the basket may be replaced with another type of tool, such as a grasping assembly or biopsy device; the device may be used without an endoscope; objects other than those mentioned or in different locations in the body may be retrieved, etc.

Thus the scope of the invention should be determined by the appended claims and their legal equivalents, rather than by the examples given.

What is claimed is:

1. A medical retrieval device for extracting an object from the urinary or biliary system comprising:
a cable comprising a sheath and a drive wire, said drive wire extending through said sheath, said cable having a proximal end and a distal end,
an object entrapping assembly located at the distal end of said cable for entrapping said object, said sheath operable for the collapsing of said object entrapping assembly when withdrawn into said sheath, and
a handle assembly located at the proximal end of said cable, said handle assembly for enabling the movement of said object entrapping assembly into and out of said sheath, said handle assembly comprising at least a longitudinally stationary portion and a sliding portion, part of an exposed area of said sliding portion has a thumb pad that the operator would normally use to move said sliding portion, longitudinal movement of said sliding portion relative to said longitudinally stationary portion causes actuation of said object entrapping assembly into or out of said sheath, at least some portion of said thumb pad will overlap said longitudinally stationary portion at some point during the actuation of said medical retrieval device, said handle assembly further comprising a cable protection means for resisting kinking and bending of said cable apart from a flexible strain relief member surrounding the cable adjacent the handle assembly,
whereby said cable protection means provides increased kink and bend protection for said cable.

2. The medical retrieval device of claim 1 wherein the length of said longitudinally stationary portion is approximately ten centimeters or greater.

3. The medical retrieval device of claim 1 wherein said cable protection means comprising a weight of said handle assembly of approximately seven grams or less.

4. The medical retrieval device of claim 1 wherein the width of said longitudinally stationary portion is approximately 1.2 centimeters or greater for at least 25% of the length of said longitudinally stationary portion.

5. The medical retrieval device of claim 1 wherein said longitudinally stationary portion comprising a distal end and a proximal end, a hole is located at said distal end for accepting said cable, said hole is sized to allow radial movement of said cable by at least 0.5 mm at said distal end of longitudinally stationary portion

6. The medical retrieval device of claim 1 wherein said weight of said handle assembly is between two and six grams.

7. The medical retrieval device of claim 1 wherein said longitudinally stationary portion is substantially formed of a thermoplastic having a specific gravity of 0.9 to 1.5.

8. The medical retrieval device of claim 1 further including a series of openings extending longitudinally down said longitudinally stationary portion.

9. The medical retrieval device of claim 1 wherein length of said cable is about 50 centimeters or greater and at least some of said cable has an outside diameter of approximately one millimeter or less.

10. A medical retrieval device for extracting an object from the urinary or biliary system comprising:
a cable comprising a sheath and a drive wire, said drive wire extending through said sheath, said cable having a proximal end and a distal end,
an object entrapping assembly located at the distal end of said cable for entrapping said object, said sheath operable for the collapsing of said object entrapping assembly when withdrawn into said sheath, and
a handle assembly located at the proximal end of said cable, said handle assembly for enabling the movement of said object entrapping assembly into and out of said sheath, said handle assembly comprising at least a longitudinally stationary portion and a sliding portion, part of an exposed area of said sliding portion has a thumb pad that the operator would normally use to move said sliding portion, longitudinal movement of said sliding portion relative to said longitudinally stationary portion causes actuation of said object entrapping assembly into or out of said sheath, said longitudinally stationary portion contains a plurality of openings in series in said longitudinally stationary portion,
whereby said openings improve accessibility for sterilization and aeration and reduce weight for increased kink and bend protection for said cable.

11. The medical retrieval device of claim 10 wherein said plurality of openings comprises at least ten openings that pass completely through said longitudinally stationary portion.

12. The medical retrieval device of claim 10 wherein said plurality of openings comprises at least two series of openings extending longitudinally down said longitudinally stationary portion.

13. The medical retrieval device of claim 10 wherein the weight of said handle assembly is seven grams or less.

14. The medical retrieval device of claim 10 wherein at least one of said plurality of openings has an area of approximately 2 cm² or less and the openings pass completely through said longitudinally stationary portion.

15. A medical retrieval device for extracting an object from the urinary or biliary system comprising:
a cable comprising a sheath and a drive wire, said drive wire extending through said sheath, said cable having a proximal end and a distal end,
an object entrapping assembly located at the distal end of said cable for entrapping said object, said sheath operable for the collapsing of said object entrapping assembly when withdrawn into said sheath, and
a handle assembly located at the proximal end of said cable, said handle assembly for enabling the movement of said object entrapping assembly into and out of said sheath, said handle assembly comprising at least a longitudinally stationary portion and a sliding portion, part of an exposed area of said sliding portion has a thumb pad that the operator would normally use to move said sliding portion, longitudinal movement of said sliding portion relative to said longitudinally stationary portion causes actuation of said object entrapping assembly into or out of said sheath, said longitudinally stationary portion comprising a distal end and a proximal end, a hole is located at said distal end for accepting said cable, said hole is sized to allow radial movement of said cable by at least 0.5 mm at said distal end of longitudinally stationary portion,
whereby improved flexibility at said distal end from the oversized cable hole provides increased kink and bend resistance for said cable.

16. The medical retrieval device of claim 15 wherein length of said cable is about 50 centimeters or greater and at least some of said cable has an outside diameter of approximately one millimeter or less.

17. A medical retrieval device for extracting an object from the urinary or biliary system comprising:
a cable comprising a sheath and a drive wire, said drive wire extending through said sheath, said cable having a proximal end and a distal end,
an object entrapping assembly located at the distal end of said cable for entrapping said object, said sheath operable for the collapsing of said object entrapping assembly when withdrawn into said sheath, and
a handle assembly located at the proximal end of said cable, said handle assembly for enabling the movement of said object entrapping assembly into and out of said sheath, said handle assembly comprising at least a longitudinally stationary portion and a sliding portion, part of an exposed area of said sliding portion has a thumb pad that the operator would normally use to move said sliding portion, longitudinal movement of said sliding portion relative to said longitudinally stationary portion causes actuation of said object entrapping assembly into or out of said sheath, at least some portion of said thumb pad will overlap said longitudinally stationary portion at some point during the actuation of said medical retrieval device, said handle assembly further comprising a weight of approximately seven grams or less,
whereby said cable protection means provides increased kink and bend protection for said cable.

18. The medical retrieval device of claim 17 wherein the length of said longitudinally stationary portion is approximately ten centimeters or greater.

19. The medical retrieval device of claim 17 wherein said weight of said handle assembly is between two and six grams.

20. The medical retrieval device of claim 17 wherein said longitudinally stationary portion is substantially formed of a thermoplastic having a specific gravity of 0.9 to 1.5.

21. The medical retrieval device of claim 17 further including a series of openings extending longitudinally down said longitudinally stationary portion.

22. The medical retrieval device of claim 17 wherein length of said cable is about 50 centimeters or greater and at least some of said cable has an outside diameter of approximately one millimeter or less.
